(19) United States
(12) Patent Application Publication
(10) Pub. No.: US 2004/0096971 A1
(43) Pub. Date: May 20, 2004

(54) Thymic epithelial progenitor cells and uses thereof

(76) Inventors: Catherine Clare Blackburn, Edinburgh (GB); Craig Scott Nowell, Edinburgh (GB)

Correspondence Address: STERNE, KESSLER, GOLDSTEIN & FOX PLLC, 1100 NEW YORK AVENUE, N.W., WASHINGTON, DC, 20005, US

(21) Appl. No.: 10/451,420

(22) PCT Filed: Dec. 24, 2001
(86) PCT No.: PCT/GB01/05780

(30) Foreign Application Priority Data
Dec. 22, 2000 (GB) 0031507.7
Apr. 30, 2001 (GB) 0110583.2

(51) Int. Cl. C12N 5/08; C12N 15/85

(52) U.S. Cl. 435/455; 435/366

(57) ABSTRACT

A method for improving the viability of a population of isolated thymic epithelial progenitor cells (TEPCs) comprises contacting the cells, or one or more ancestors thereof, with at least one viability promoting agent A TEPC line is provided and used for restoring or anhancing thymic function and for generation of T cells from haematopoietic stem cells.

The present invention relates to thymic epithelial progenitor cells (TEPCs) and more particularly to materials and methods for producing, maintaining and using said cells, e.g. for therapeutic purposes.

The thymus is the principal site of T-cell development, providing the microenvironments required to support T-cell differentiation and repertoire selection (Miller, J. F. A. P. Lancet 2: 748-749, (1961); Anderson, G. Annu. Rev. Immunol. 14: 73-99 (1996)). These unique processes are dependent on the thymic stroma (Boyd, R. L. Immunol. Today 14:445-459 (1993)). This comprises a highly ordered, three-dimensional network of thymic epithelial cells interspersed with non-epithelial stromal cell-types and is organized into two main compartments, the cortex and the medulla, which each contain several distinct epithelial subpopulations (Boyd (1993); Anderson (1996); Van Ewijk, W. Semin. Immunol. 11: 57-64 (1999)). The different thymic epithelial cell-types are thought to provide specific molecular niches required for different stages of thymocyte differentiation and maturation, as T-cell development requires interactions with multiple epithelial subpopulations (Anderson, G. Immunol. Today 20:463468 (1999); Ge, Q. Int. Immunol. 12:1127-1133 (2000)).

During embryogenesis, contributions from the 3^{rd} pharyngeal pouch endoderm, neural crest-derived mesenchyme and possibly the 3^{rd} pharyngeal cleft ectoderm are required for formation of the thymic primordium, which is first present as a discrete organ at day 12.5 of murine embryonic development (E12.5) (Manley, N. Semin. Immunol. 12: 421-428 (2000)). Sequential reciprocal interactions between thymocytes and immature thymic epithelium are subsequently needed to establish proper organization and function of the cortical and medullary compartments (Van Ewijk (1999); Ritter, M. A. Immunol. Today 14:462-469(1993)). Vascularization may also be required for maturation of the medulla (Anderson, M. Int. Immunol. 12: 1105-1110 (2000)), and may constitute a developmental checkpoint associated with gain of competence to support thymocyte maturation (Fairchild, P. J. Eur. J. Immunol. 30: 1948-1956 (2000)).

It is desirable to have in vitro means of developing T cells and their progenitors. Thymic epithelial complexity has proved a stumbling block for attempts to generate T-cells in vitro, and this is currently possible only in organ cultures based on ex vivo thymic tissue (Hare, K. J., Jenkinson, E. J. & Anderson G. In vitro models of T cell development. Semin. Immunol. 11,3-12 (1999); Poznansky, M. C. et al. Efficient generation of human T cells from a tissue-engineered thymic organoid. Nature Biotech. 18, 729-734 (2000)). Improvements in this area are highly desirable, since the ability to generate T-cells efficiently in vitro would impact significantly on clinical outcome in treatments of post-chemoradiotherapy leukaemia and cancer patients and organ transplant recipients (Eisner, Y. & Martelli, M. F. Tolerance induction by ‘megadose’ transplants of CD34+stem cells; a new option for leukemia patients without an HLA-matched donor. Curr. Opinion. Immunol. 12, 536-541 (2000), Slavin, S. New Strategies for bone marrow transplantation. Curr. Opinion. Immunol 12, 542-551 (2000).

Based on an analysis of wild-type thymic epithelial cells and of cells committed to thymic epithelial lineages but unable to express Foxn1 (formerly whn/Hfh11) (Kaestner, K. H. Genes Dev. 14: 142-146 (2000)), a transcription factor (Nehls, M. Science 272:886-889 (1996)) required cell-autonomously for development of all mature thymic epithelial subpopulations (Blackburn, C. C. Proc. Natl. Acad. Sci. USA 93: 5742-5746 (1996); the present inventors have shown that two monoclonal antibodies, MTS20 and MTS24, identify a population of progenitor cells within the murine thymic primordium. When purified from thymic primordia isolated from wild-type E 12.5 mouse embryos and grafted under the kidney capsule of recipient mice, these MTS20⁺/24⁺ cells differentiated into cortical and medullarythymicepithelial cell-types, attracted lymphoid progenitors and supported thymocyte differentiation. Moreover, they conferred thymus function on congenitally athymic recipient mice. The corresponding MTS20⁻/24⁻ population, which includes cells expressing markers associated with mature cortical epithelium, could not fulfil these functions. MTS20⁺/24⁺ expression therefore identifies a thymic epithelial progenitor cell-type or types (TEPC), a thymic progenitor- or stem cell capable of differentiating into both cortical or medullary thymic epithelial cells, sufficient to form a functional thymus in vivo.

In the present application, the inventors address the need for a method to allow the generation of T cells and disclose uses of TEPCs and compositions containing them. In vivo they may be used to restore thymic function in athymic individuals, e.g. in patients suffering from DiGeorge syndrome or from the human “nude” condition or to augment or customise thymus function eg. to promote allograft acceptance eg. in bone marrow and organ transplant recipients. In vitro they may be used to generate artificial thymi, thereby enabling the generation of mature T-cell populations from haematopoietic stem cells (HSCs) and/or lymphoid progenitors. Such artificial thymi can be customised for particular purposes, e.g. for the in vitro or in vivo generation of T-cell populations which are tolerant to the tissues of two or more individuals. These and other uses of TEPCs provide important aspects of the present disclosure.

Based on their in vivo properties, TEPCs are an ideal material for use in transplantation therapy or for in vitro thymi generation. They are an expandable cell-type, capable of producing all major mature thymic epithelial sub-populations. In vitro however they have proven difficult to maintain in culture. The present disclosure therefore provides materials and methods for enriching TEPC populations, for improving the viability of an isolated TEPC, for expanding a population of TEPCs in vitro, and for causing or allowing TEPCs to differentiate into cortical and medullary thymic epithelial cell-types to generate a functional thymus in vitro or in vivo. This strategy circumvents both ethical and practical issues surrounding the use in culture or for transplantation of cells obtained directly from human fetal tissue. In particular, each fetus provides only a small number of cells, insufficient for clinical purposes.

In a first aspect, the invention provides a method for improving the viability of a population of isolated thymic epithelial progenitor cells (TEPCs), which method comprises contacting the cells, or one or more ancestors thereof, with at least one viability promoting agent.

By “improving the viability of a population of isolated thymic epithelial progenitor cells (TEPCs)” is meant that the rate of decline of the number of viable TEPCs in the population is reduced. This may include the number of viable TEPCs in the population being maintained at a substantially constant level, or the number of viable TEPCs in the population being increased over time.

By “isolated TEPCs” is meant that the TEPCs are not associated with at least one cell-type with which TEPCs are normally associated in vivo under physiological conditions. The TEPCs may be isolated away from one or more of: mesenchymal cells, T-cell progenitors, thymocytes, vascular endothelium, differentiated thymic epithelial cells, bone-marrow derived thymic stromal cells.

In preferred embodiments, the one or more viability promoting agents induces or enhances TEPC replication. In this way, the decline in the viability of the TEPC population as a whole may, at least in part, be slowed, arrested or reversed, by the production of daughter cells from an original group of TEPCs. If a replicating population of cells approaches confluence, then the population may be sub-divided into two or more daughter populations. Each population may be diluted in a suitable medium, as discussed elsewhere herein.

The one or more viability promoting agents may be protein, polypeptide, glycoprotein, proteoglycan, carbohydrate, oligosaccharide, polysaccharide, nucleotide, oligonucleotide or nucleic acid in nature. The agent may be selected from the group consisting of a hormone, growth factor, cytokine, steroid, interferon, colony stimulating factor, extracellular matrix material. It may be produced by a specific cell-type or cell-types, and may be a cell surface agent and/or an agent sereted in to the culture supernatant of those cells.

Specific examples of suitable agents include insulin-like growth factor 1 IGF-1, epidermal growth factor EGF, insulin, hydrocortisone, transferrin, high density lipoprotein (HDL), bone morphogenetic protein (BMP2)2, (BMP)4 and (BMP)7 noggin, fibroblast growth factor 1 (Fgf1), Fgf2, Fgf3, Fgf8, and sonic hedgehog (shh).

The one or more viability promoting agents may be added continually or periodically to the TEPC population. Alternatively, there may be a single, initial period of exposure to the one or more agents, which period can involve a single addition of the one or more agents or a plurality of successive additions. Where a TEPC population is contacted with one or more viability promoting agents on a number of consecutive occasions, the agent or agents added on each occasion may be different from those added on a previous occasion.

Where the TEPC population is subjected to a single, initial period of exposure, the one or more agents may cause the cells to undergo a long-term physiological change. That change may enable the viability of the population of TEPCs to be substantially improved without the need for a subsequent addition of any further viability promoting agents.

In certain embodiments of the invention, the one or more viability promoting agents may cause a change in the genotype of at least one TEPC in the population. This change in genotype may improve the viability of the TEPC, e.g. by transforming the TEPC into an immortalized or reversibly immortalized state. In this connection, the one or more viability promoting agents may include at least one polynucleotide.

The polynucleotide may be part of a vector which may be plasmid or viral or artificial chromosome. Suitable vectors can be chosen or constructed, containing appropriate regulatory sequences, e.g. promoter sequences, terminator fragments, polyadenylation sequences, enhancer sequences. Vectors may contain selectable marker genes and other sequences as appropriate.

Marker genes such as antibiotic resistance or sensitivity genes, or fluorescent- or epitope-tagged proteins may be used in identifying clones containing nucleic acid of interest, as is well known in the art. Clones may also be identified or further investigated by binding studies, e.g. by Southern blot hybridisation.

Inside the TEPC, the nucleic acid comprising the polynucleotide may exist as an isolated extra-genomic sequence, or it may integrate, preferably stably, into the host cell genome. As an isolated sequence, it may be capable of replication, e.g. as an episome or artificial chromosome. Integration may be promoted by including in the nucleic acid sequences which promote recombination with the genome, in accordance with standard techniques. The nucleic acid may include sequences which direct its integration to a particular site in the genome where a coding sequence contained within it falls under the control of regulatory elements able to drive and/or control expression of that sequence in the TEPC.

Methods for introducing nucleic acid into cells are well known to those skilled in the art and include e.g. ballistic bombardment, calcium phosphate transfection, DEAE-Dextran, electroporation, liposome-mediated transfection and transduction using retrovirus or other vectors. Suitable vectors include adenovirus, papovavirus, vaccinia virus, herpes virus and retroviruses. Disabled virus vectors may be produced in helper cell lines in which genes required for production of infectious viral particles are expressed. Suitable helper cell lines are well known to those skilled in the art. By way of example, see: Fallaux, F. J., et al., (1996) Hum Gene Ther 7(2), 215-222; Willenbrink, W., et al., (1994) J Virol 68(12), 8413-8417; Cosset, F. L., et al., (1993) Virology 193(1), 385-395; Highkin, M. K., et al., (1991) Poult Sci 70(4), 970-981; Dougherty, J. P., et al., (1989) J Virol 63(7), 3209-3212; Salmons, B., et al., (1989) Biochem Biophys Res Commun 159(3), 1191-1198; Sorge, J., et al., (1984) Mol Cell Biol 4(9), 1730-1737; Wang, S., et al., (1997) Gene Ther 4(11), 1132-1141; Moore, K. W., et al., (1990) Science 248(4960),1230-1234; Reiss, C. S., et al., (1987) J Immunol 139(3), 711-714. Helper cell lines are generally missing a sequence which is recognised by the mechanism which packages the viral genome. They produce virions which contain no nucleic acid. A viral vector which contains an intact packaging signal along with the gene or other sequence to be delivered is packaged in the helper cells into infectious virion particles, which may then be used for gene delivery to the TEPC.

It will be apparent to the skilled person that the particular choice of method used to introduce nucleic acid into the TEPC is not essential to or a limitation of the invention.

A viability promoting agent which consists of or comprises a polynucleotide may include the whole or part of an open reading frame (ORF). The ORF may be operably-linked to a promoter which drives its expression in TEPCs. “Operably linked” means joined as part of the same nucleic acid molecule, suitably positioned and oriented for transcription to be initiated from the promoter.

The polynucleotide may be placed under the control of an externally inducible gene promoter to place it under the control of the user. The term “inducible” as applied to a promoter is well understood by those skilled in the art. In essence, expression under the control of an inducible promoter is “switched on” or increased in response to an applied stimulus. The nature of the stimulus varies between promoters. Some inducible promoters cause little or undetectable levels of expression (or no expression) in the absence of the appropriate stimulus. Whatever the level of expression is in the absence of the stimulus, expression from any inducible promoter is increased in the presence of the correct stimulus. An example of an inducible promoter is the Tetracyclin ON/OFF system (Gossen, et al., (1995) Science, 268, 1766-1769) in which gene expression is regulated bytetracyclin analogs. Expression of the polynucleotide may also be controlled or regulated by one or more additional elements in the transformed nucleic acid, e.g. by an enhancer.

A viability promoting agent which consists of or comprises a polynucleotide may include a promoter which is not operably linked to an ORF. It may include an enhancer or other transcriptional control sequence. The nucleic acid may be integrated into the genome of the TEPC so as to induce, increase, inhibit or prevent expression of a neighbouring coding sequence. The nucleic acid may include sequences which direct its integration to a particular site in the genome, .g. by virtue of their homology with sequences surrounding that site. A polynucleotide viability promoting agent may also comprise an antisense sequence or a ribozyme, or DNA encoding such a sequence. The antisense RNA or ribozyme may interfere with a cell-cycle checkpoint, thereby resulting in immortalisation of the host cell.

Expression of a polypeptide, whether derived from either part of the transformed nucleic acid or from the TEPC genome, may be constitutive or inducible. Induction may require the addition of one or more additional agents to the TEPC, e.g. simultaneously with or subsequent to the contact of the TEPC with the viability promoting agent.

Examples of polynucleotides which may be used as viability promoting agents include oncogenes and transposable elements. A specific example is the SV40 T antigen.

An oncogene may transform the TEPC into an immortalized state. It may be conditionally inactivatable such that a reversible immortalisation may be achieved. An immortalizing oncogene which is inactive at the body temperature of a human patient may be used. Inactivation of the oncogene reduces the risk of tumor formation when TEPCs, or descendants thereof, are introduced into the patient during a method of therapy. Immortalizing oncogenes may also be removed from TEPCs, or descendants thereof, prior to the introduction of such cells into a patient. Removal of oncogenes may employ the Cre-LoxP system (Westerman, K. A. et al Proc. Natl. Acad Sci. USA 93, 8971 (1996)).

Many known techniques and protocols for manipulation of nucleic acid, for example in preparation of nucleic acid constructs, mutagenesis, sequencing, introduction of nucleic acid into cells and gene expression, and analysis of proteins, are described in detail in Current Protocols in Molecular Biology, Ausubel et al. eds., John Wiley & Sons, (1992) and Molecular Cloning: a Laboratory Manual: 3rd edition, Sambrook and Russell, 2001, Cold Spring Harbor Laboratory Press.

Viability promoting agents for use in the present invention may easily be identified on the basis of routine techniques well known to those skilled in the art. By way of example, a freshly isolated population of TEPCs may first be sub-divided into two populations: a “test” population and a “control” population. One or more test agents are then added to the “test” population and the proportion of viable cells retaining the TEPC phenotype in each population is determined (measurement at t=0). After a predetermined period of time (t=1), during which time the test agent(s) may again be added to the test population, the proportion of viable cells retaining the TEPC phenotype in the population is again determined. The change in the proportion of viable cells retaining the TEPC phenotype in each population from t=0 to t=1 is calculated.

If the reduction in the proportion of viable cells retaining the TEPC phenotype within the test population is less than that in the control population, then the one or more test agents is/are suitable candidates for viability promoting agents. The proportion of viable cells in each population may be determined at time intervals subsequent to t=1, so as to better assess the ability of the one or more test agents to promote the viability of TEPCs.

The proportion of viable cells in a TEPC population may easily be determined using known techniques for assessing cell viability. By way of example, a sample of the TEPC population may be taken, and the number of viable cells in the sample determined, e.g. using a microscope, e.g. using trypan blue, by counting the number of viable cells, in the whole or part of the sample, optionally after dilution. The proportion of viable cells may be determined in relation to e.g.: (a) the total number of cells in the sample; or (b) the total volume of the sample. The proportion of viable cells retaining the TEPC phenotype may be determined in flow cytometric or immunocytochemical analysis.

Where a conditionally active test agent is used, e.g. a conditionally active oncogene, a difference in the ability of the agent to improve or maintain the viability of the contacted TEPC between the permissive and non-permissive conditions indicates that the test agent is a candidate for a viability promoting agent.

In certain embodiments of the invention, the isolated TEPC which is contacted with the one or more viability promoting agents, is cultured under conditions which inhibit differentiation of the TEPCs into e.g. cortical or medullary thymic epithelial cells. The TEPCs may be cultured in the absence of e.g. mesenchymal cells and/or T-cell progenitors and/or thymocytes. Such cell-types have been shown to drive the production of cortical and medullary thymic epithelial cell-types in thymic organ cultures.

Differentiation of the TEPCs, e.g. into cortical or medullary thymic epithelial cells, may also be arrested by the introduction into TEPCs, or into ancestors thereof, of a nucleic acid sequence.

The nucleic acid sequence may inhibit differentiation by promoting proliferation. Such a nucleic acid sequence may affect the control of the cell cycle. It may be an oncogene. The nucleic acid sequence may comprise one or more control elements which, upon induction or inhibition, will permit the differentiation of the host TEPC to proceed.

The nucleic acid which arrests differentiation of TEPCs may cause suppression or ablation of Foxn1 expression: as discussed elsewhere herein, TEPCs cannot differentiate into mature thymic epithelial cell sub-populations without Foxn1 expression. The nucleic acid may cause conditional suppression of Foxn1 expression such that under permissive conditions, differentiation of the TEPC may proceed. It may for example comprise: (i) an inducible promoter responsive to the addition of one or more agents; and (ii) targeting sequences which direct integration of the promoter into the TEPC genome so as to functionally replace the wild-type Foxn1 promoter; or (iii) a Foxn1 transgene in which the Foxn1 regulatory elements are replaced by an inducible promoter responsive to the addition of one or more agent. The transgene may be randomly integrated in to a TEPC genome which is Foxn1^{-/-}, or a wild-type TEPC genome which is then backcrossed onto a Foxn1^{-/-} background, e.g. a nude mouse. Alternatively it may comprise (i) targeting sequences which direct integration of a modified Foxn1 gene into the TEPC genome so as to functionally replace the wild-type gene; or (ii) sequences which permit random integration of a modified Foxn1 transgene, including Foxn1 regulatory regions, into the TEPC genome which again may be Foxn1^{-/-}, or may be a wild-type TEPC genome which is then backcrossed onto a Foxn1^{-/-} background. The Foxn1 transgene may be modified such that Foxn1 expression is reversibly ablated. This may be achieved by the introduction of a stop cassette into the Foxn1 gene, downstream of the Foxn1 transcriptional start. The stop cassette may be excised from the genome to allow Foxn1 expression, and hence TEPC differentiation, to proceed. This may be achieved using the Cre-LoxP system, or the Flp recombinase system, or other recombinase systems. Alternatively Foxn1 expression may be reversibly ablated by the introduction into the TEPC genome of nucleic acids comprising constructs designed to express antisense Foxn1 RNA, constructs designed to express Foxn1-specific ribozyme, constructs encoding a dominant negative Foxn1 protein, or constructs encoding a protein or agent capable of sequestering Foxn1 within the cell and thus rendering it inactive.

The integration of nucleic acid at a specific point in the TEPC genome may be achieved by sequences promoting homologous recombination. Alternatively, the nucleic acid may be inserted as a randomly integrated transgene. Materials and methods for transforming TEPCs with nucleic acid are described elsewhere herein.

Differentiation of TEPCs to cortical or medullary thymic epithelial cell-types may be detected by any of the methods described elsewhere herein.

In certain embodiments of the invention, the isolated TEPC is contacted with the one or more viability promoting agents, and/or is cultured adjacent to, or in medium conditioned by, explant cultures from one or more tissues selected from the group consisting of foetal heart tissue and tissue from branchial arch, e.g. whole branchial arch or branchial arch ectoderm. These tissues are adjacent the thymic primordium in the embryonic state. The tissues may be kept apart from the isolated TEPC by an appropriate membrane or filter. The isolated TEPC may be contacted in the presence of one or more growth factors expressed in these tissues. The isolated TEPC may be cultured on irradiated feeder cells, selected from the group consisting of fibroblast cells, or embryonic thymic epithelial cells, or branchial arch cells. The fibroblast cells may be transfected such that they express gene or genes encoding a specific growth factor.

The isolated TEPC may originally be derived from an embryo, e.g. from an embryo which has developed at least as far as murine E11.25, or equivalent stages in other mammals. In one protocol, the cells in an embryonic thymus are dissociated from one another and the TEPCs isolated by flow cytometry. Fluorescence Activated Cell Sorting (FACS) may be used to identify and partition TEPCs from other thymic cell-types: the monoclonal antibodies MTS20 and MTS24 may be used. Alternatively or in addition, TEPCs may be isolated from other thymic cell populations by size selection using light scatter parameters. Isolation of TEPCs by size selection avoids the potentially activating effects of monoclonal antibodies and thereby helps to maintain the TEPCs in an undifferentiated state. Alternatively or in addition, TEPC may be derived in vitro from multipotent ancestor cells, e.g. ES cells.

The isolated TEPCs may originate from non-human transgenic or chimaeric mammalian embryos, i.e. embryos containing foreign or heterologous nucleic acid which is absent from the corresponding wild-type cells, or human embryos. The heterologous nucleic acid may be found throughout the embryo or it may be localised to certain parts. It may contain elements which produce a phenotypic effect in a limited number of cell-types. In this way, a method of improving the viability of an isolated TEPC may comprise contacting an ancestor of the TEPC with a polynucleotide-based viability promoting agent. Materials and methods for introducing nucleic acid into cells are described elsewhere herein.

The identity of isolated cells as TEPCs may be confirmed by analysing expression by said cells of one or more of the following markers: Foxn1 (formerly whn/Hfh11), Pax-1, Pax-9, Hoxa-3, keratin 5, keratin 8, MHC Class II, epitopes reactive with MTS20 and/or MTS24.

Markers may be detected according to any method known to those skilled in the art. The detection method may employ a specific binding member capable of binding to a nucleic acid sequence encoding the marker, the specific binding member comprising a nucleic acid probe capable of hybridising with said sequence, or an immunoglobulin/antibody domain with specificity for the nucleic acid sequence or the polypeptide encoded by it. A specific binding member has a particular specificity for the marker and in normal conditions binds to the marker in preference to other species. Alternatively, a specific mRNA for the marker may be detected by its binding to specific oligonucleotide primers and amplification in e.g. the polymerase chain reaction (Current Protocols in Molecular Biology, Ausubel et al. eds., John Wiley & Sons, (1992) and Molecular Cloning: a Laboratory Manual: 3rd edition, Sambrook and Russell, 2001, Cold Spring Harbor Laboratory Press; Antibodies: A Laboratory Manual: Harlow, E. and Lane, D., 1988, Cold Spring Harbor Laboratory Press).

Binding or interaction may be determined by any number of techniques known in the art, qualitative or quantitative. Interaction between the specific binding member and the marker may be studied by labeling either one with a detectable label and bringing it into contact with the other which may have been immobilized on a solid support, e.g. by using a secondary antibody bound to a solid support. Interaction between a specific binding member and a marker expressed on a cell surface may be detected by flow cytometry.

Flow cytometric techniques are by now well established. Detailed protocols are compiled in several recent compendia, including Flow Cytometry: A Practical Approach, 2^{nd} ed. M. G. Ormerod (ed.), Oxford University Press (1997); Handbook of Flow Cytometry Methods, J. Paul Robinson (ed.), John Wiley & Sons (1993); Current Protocols in Cytometry, J. Paul Robinson (ed.), John Wiley & Sons (1997 with updates). Fluorescent labels for conjugation to specific binding members include e.g. FITC (fluorescein isothiocyanate), PE (phycoerythrin), PerCP (peridinium chlorophyll protein), APC (allophycocyanin), PE-CY5 tandem fluorophore (phycoerythrin-cyanine 5 tandem resonance energy transfer fluorophore; Cychrome) or PerCP-CY5.5 tandem fluorophore.

Other materials and methods for isolating TEPCs may include magnetic sorting (Johansson, et al., (1999) Cell, 96, 25-34) and/or lysing non-TEPC cells, e.g. by labelling them with antibodies and exposing them to complement. Cortical thymic epithelial cells may be labelled with 4F1 and both cortical and medullary thymic epithelial cells may be labelled with appropriate MHC Class II-specific antibodies.

Once isolated from a thymic cell population, the TEPCs are re-suspended in a suitable medium. An example of such a medium is D-valine modified DMEM (available from Sigma). Base media may be supplemented with one or more compounds selected from the group consisting of: nutritional additives, vitamins or minerals, antibiotics, antifungals and antivirals. DMEM may be supplemented with one or more of: glutamine, sodium pyruvate, non-essential amino acids, foetal calf serum, and gentamycin solution.

The TEPC populations may be maintained by seeding cells into: (i) wells coated with extra cellular matrix gel; (ii) uncoated wells; (iii) wells coated with defined extracellular matrix components (e.g. laminin, collagen); (iv) wells coated with gelatin; or wells coated with irradiated feeder cells, e.g. irradiated fibroblasts, e.g. irradiated E12.5 thymus cells. The one or more viability promoting agents may be contacted with the population of isolated TEPCs before or after the seeding of the cells.

In a further aspect, the present invention provides a TEPC which has been maintained in a viable, undifferentiated state, by any one of the methods disclosed herein.

A related aspect is a TEPC containing heterogeneous nucleic acid which promotes the viability of the TEPC in vitro. The nucleic acid may be of the type described elsewhere herein, e.g. a transforming oncogene, which may be conditionally inactivatable.

The invention further extends to a pharmaceutical composition, medicament, drug or other composition comprising a TEPC according to the present invention, use of such a TEPC or composition in a method of medical treatment, a method comprising administration of such a TEPC or composition to a patient, e.g. to restore thymic function in an athymic individual, e.g. for the treatment of DiGeorge Syndrome or the human “nude” condition, or to augment or customise thymus function and promote allograft acceptance e.g. in bone marrow- and organ transplant patients, use of a TEPC of the invention in the manufacture of a medicament for administration to a patient, e.g. to an athymic patient for the restoration of thymic function in DiGeorge Syndrome or the human “nude” condition, or to augment or customise thymus function and promote allograft acceptance e.g. in bone marrow- and organ transplant patients, and a method of making a pharmaceutical composition comprising admixing such a population with a pharmaceutically acceptable excipient, vehicle or carrier.

Pharmaceutical compositions according to the present invention, and for use in accordance with the present invention, may comprise, in addition to the population of TEPCs, a pharmaceutically acceptable excipient, carrier, buffer, preservative, stabiliser, anti-oxidant or other material well known to those skilled in the art. Such materials should be non-toxic and should not interfere with the activity of the TEPCs. The precise nature of the carrier or other material will depend on the route of administration.

Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

The composition may be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride, Ringer's Injection, or Lactated Ringer's Injection. Administration of a composition in accordance with the present invention is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of the condition being treated. Prescription of treatment, e.g. decisions on dosage etc, is within the responsibility of general practitioners and other medical doctors.

A composition may be administered alone or in combination with other treatments, either simultaneously or sequentially dependent upon the condition to be treated. TEPCs may be implanted into a patient by any technique known in the art.

An isolated TEPC maintained in accordance with the present invention may be allowed or caused to differentiate into a mature thymic epithelial cell-type, e.g. into a cortical or medullary thymic epithelial cell. Such differentiation may occur in vitro or in vivo after removal of an immortalizing agent and/or removal of a differentiaton blocking agent as appropriate. A pharmaceutical composition, medicament or drug of the invention may therefore comprise, in addition to the isolated TEPC, one or more factors which promote the differentiation of the TEPC, e.g. into a cortical thymic epithelial cell or a medullary thymic epithelial cell. Such factors may be supplied by or derived from a mesenchymal cell and/or T-cell progenitor or thymocyte.

In a further aspect, the invention provides an in vitro method of generating a mature thymic epithelial cell from a TEPC, which method comprises culturing the TEPC, under conditions which promote removal of an immortalizing agent and/or removal of a differentiaton blocking agent if appropriate, in the presence of one or more factors supplied by or derived from a mesenchymal cell and/or T-cell progenitor or thymocyte which promote differentiation of the TEPC into a mature thymic epithelial cell-type, e.g. a cortical or medullary thymic epithelial cell.

If a population of TEPCs is used, a majority of the TEPCs may be caused or allowed to adopt mature thymic epithelial fate. In preferred embodiments, more than 60%, more than 70%, more than 80%, more than 90% of the TEPCs differentiate into mature thymic epithelial cells. Specific factors that may cause TEPC to differentiate are the Wnt family of proteins (Wnt1, Wnt4, Wnt5b, Wnt10b), the BMP family of proteins (BMP2, BMP4), the Fgf family of proteins (Fgf7, Fgf8, Fgf10).

The method may comprise culturing the TEPC, e.g one from which the immortalizing agent and/or differentiaton blocking agent have been removed, with:

(i) a mesenchymal cell and/or T-cell progenitor or thymocyte; and/or

(ii) one or more factors secreted from mesenchymal cells, thymocytes, T-cell progenitors, T-cells or vascular endothelium.

It may comprise culturing the TEPC with a fibroblast or bone marrow cell.

The mesenchymal cells, T-cell progenitors, fibroblasts and bone marrow cells may each be derived from a cell line. Cell lines provide a homogeneous cell population.

The method may comprise detecting differentiation of the TEPC to either a cortical or thymic epithelial cell. Cortical thymic epithelial cells may be identified by their expression of epitopes reactive with the monoclonal antibody 4F1 and/or their expression of MHC Class II. Medullary thymic epithelial cells may be identified by their expression of epitopes reactive with MTS10 and/or their expression of MHC Class II. Binding or interaction of 4F1 and/or MTS10 and/or MHC Class II with cortical and/or medullary thymic epithelial cell-types may be detected in accordance with any of the methods described elsewhere herein. Differentiation may also be detected by observing changes in cell morphology, e.g. by microscopy, and/or by gene expression analysis.

The method may comprise the step of separating cortical and/or medullary epithelial cells from a culture of TEPCs which have undergone differentiation to a mature thymic epithelial cell-type. Such separation may employ FACS, e.g. using fluorescently-labeled 4F1 and/or anti MHC Class II and/or adhering said cells to an immunoadsorbent, e.g. to a solid support having 4F1 and anti MHC Class II immobilized thereon. Alternatively, the method may comprise separating undifferentiated TEPCs from a culture containing TEPCs which have undergone differentiation to a mature thymic epithelial cell-type. Such separation may employ FACS, e.g. using MTS20 and MTS24, e.g. using a marker introduced into the genome of the TEPC such that it is expressed in TEPC but not mature thymic epithelial cell-types, or such that the marker is removed upon removal of the immortalizing agent and/or the differentiation blocking agent. TEPC were purified with both MTS20 and MTS24, or either antibody alone. Purified TEPC may express the determinants recognised by either or both MTS20 or MTS24.

In various further aspects, the invention extends to a mature thymic epithelial cell produced by a method of the invention, and to a pharmaceutical composition, medicament, drug or other composition comprising such a cell. The invention also extends to the use of such a cell or composition in a method of medical treatment, to a method comprising administration of such a cell or composition to a patient, e.g. to restore thymic function in an athymic individual, e.g. for the treatment of DiGeorge Syndrome or the human “nude” condition, or to augment or customise thymus function and promote allograft acceptance e.g. in bone marrow- and organ transplant patients, to the use of such a cell in the manufacture of a medicament for administration to a patient, e.g. to an athymic patient for the restoration of thymic function in DiGeorge Syndrome or in the human “nude” condition, or to augment or customise thymus function and promote allograft acceptance e.g. in bone marrowand organ transplant patients, and to a method of making a pharmaceutical composition comprising admixing such a cell with a pharmaceutically acceptable excipient, vehicle or carrier.

The formulation and administration of pharmaceutical conditions is described elsewhere herein.

TEPCs and/or their progeny, cortical and medullary thymic epithelial cells, may be used to provide a thymic function, either in vivo or in vitro. Provision of a thymic function in vitro or in vivo may require removal of an immortalizing agent and/or an agent which blocks differentiation of TEPC from the TEPC. Provision of a thymic function in vivo requires transplantation of the cells into a patient.

For an artificial thymus in vitro, the cells are cultured in a nutritive medium which may additionally comprise one or more other cell-types, e.g. non-epithelial cells of the thymic stroma, mesenchymal cells, cells of the vascular endothelium, haematopoietic stem cells/lymphoid progenitor cells. The cells may be grown on a solid support matrix. Production of a functional artificial thymus may be detected by the ability of the thymus to cause differentiation of haematopoietic stem cells (HSCs) and/or lymphoid progenitor cells to mature CD4⁺ or CD8⁺ T cells. Mature T cells may be detected using labeled antibodies against CD4 or CD8, e.g. by using microscopy or flow cytometry, as described elsewhere herein.

In a further aspect, the invention therefore provides a method of generating an artificial thymus in vitro. The method comprises providing a population of cortical and medullary thymic epithelial cells, which population has been obtained by causing or allowing differentiation of a population of isolated TEPCs.

The method may comprise inducing said differentiation by contacting the TEPCs with one or more factors supplied by or derived from mesenchymal cells, HSCs, lymphoid progenitors, thymocytes, vascular endothelial cells, or mixtures of such cells. The method may comprise co-culturing TEPCs with one or more of said cells.

The invention also extends to a method of producing mature T-cells, which method comprises contacting HSCs and/or lymphoid progenitors/thymocytes, with an artificial thymus of the invention. HSCs and lymphoid progenitors may be obtained from blood or bone marrow using standard techniques well known to those skilled in the art, e.g. by biopsy followed by e.g. FACS, affinity purification, using antibodies directed to appropriate cell markers. Such techniques may also be used to obtain the mature T-cells from the artificial thymus.

Mature T-cells produced by the present invention may be used to restore an immunological function of an individual whose immune system has been suppressed, e.g. with cyclosporin, e.g. by chemo- and/or radio-therapy, e.g. to reduce the likelihood of allo- orxeno-transplant rejection, orto supply an immunological function to an individual e.g. donor-derived recipient-tumour-specific T-cells, orT-cells specific for particular pathogens.

An artificial thymus may be generated from TEPCs extracted from the thymus of the intended recipient of the mature T-cells, or may be derived from multipotent cells derived from the intended recipient. In this way, the T-cells produced by the thymus may be tolerant of the tissues of the recipient. In certain embodiments of the invention, the TEPCs of the artificial thymus may be derived from two or more different individuals ortwo or more species. In this way, the mature T-cells produced by the thymus may be tolerant to the tissues of two or more individuals or species. This may have beneficial consequences if the T-cells are for use in allo or xeno-graft patients: the T-cells may be tolerant to both graft and host. A further option is to establish a bank of cells covering a range of immunological compatibilities from which an appropriate choice can be made for an individual patient. TEPCs cells derived from one individual may also be altered to ameliorate rejection when they or their progeny are introduced into a second individual. By way of example, one or more MHC alleles in a donor cell may be replaced with those of a recipient, e.g. by homologous recombination, or augmented with those of a recipient, or donor e.g. by additive transgenesis.

Further aspects of the present invention include a T-cell produced by the TEPC-derived artificial thymus, and a composition, medicament or drug containing such a T-cell. The invention also provides the use of such a T-cell or composition in a method of medical treatment, e.g. to restore cellular immunity, and the use of such a T-cell for the manufacture of a medicament. Formulation and administration of pharmaceutical compositions is described elsewhere herein.

In an example described in more detail below, suppression of Foxn1 activity is used to derive human TEPCs. This can be achieved by addition of Foxn1-antisense oligonucleotides, or viral or liposomal delivery of antisense-Foxn1 RNA or anti-Foxn1 ribozymes, to human embryonic thymic epithelial cultures, or by other suitable means. Alternative strategies for delivery of constructs carrying a conditionally inactivatable SV40 T antigen to primary TEPC cultures (eg retroviral delivery, adenoviral delivery) may also be used, as may strategies employing the use of constructs in which regulatory elements from TEPC or TESC-specific genes are used to control selectable marker expression.

The MTS20⁺/24⁺ cells present in adult murine thymi may be residual organ specific stem cells, thus an alternative approach can be based on reactivation of these residual TESC in adult human thymic tissue. Again, this employs contacting the cells with viability promoting agents, and/or reversible suppression of Foxn1 protein expression, and/or reversible immortalization of cells, followed by phenotypic and functional analysis.

We thus provide a TEPC population which can be propagated as clonal cell lines in vitro, and also provide efficient protocols for the derivation, growth and differentiation of TEPC lines and for the use of clonal TEPC lines to support T-cell development. Since cell lines are easily manipulable and can be expanded at will, this provides significant advantages over current in vitro T-cell differentiation strategies, which depend on the culture of ex vivo derived thymic tissue. TEPC lines may thus prove a powerful clinical tool, as they present the capacity to routinely generate in vitro large T-cell repertoires tolerant to donor-and-host tissues. These can be used to reduce infection-related morbidity in treatments requiring transplantation of T-depleted bone marrow. Additionally, T-cells of particular specificities can be expanded from these repertoires, providing an efficient means of generating, e.g. donor-derived recipient-lymphocyte-specific T-cells for donor-lymphocyte infusion protocols, e.g. pathogen-specific T-cells for transplantation into immunocompromised patients. TEPC line-based thymi can also be used in composite organ grafting protocols, which increase the rate of T-cell reconstitution and promote allo- or xeno-transplant acceptance, or to restore thymic function to athymic individuals.

Further to these benefits, the reversibly immortalized TEPC lines provide a robust, easily manipulable in vitro model for the investigation of gene function during thymus organogenesis and T-cell development. This will have application in identifying genes with important roles in these processes.

Aspects and embodiments of the present invention will now be illustrated, byway of example only and with reference to the following figures. Further aspects and embodiments will be apparent to those skilled in the art. All documents mentioned in this specification are incorporated herein by reference.

### BRIEF DESCRIPTION OF THE FIGURES

FIG. 1 shows an analysis of cells grafted under the kidney capsule of nude mice: (a) E12.5 MTS20⁺/24⁺ cell graft; (b) E12.5 MTS20⁻/24⁻ cell graft site; (c) hematoxylin and eosin stained section of graftfrom (a); (d) hematoxylin and eosin stained section of graft site from (b); (e-j) MTS20⁺/24⁺ cell graft stained for (e) cytokeratin, (f) 4F1, (g) MTS10, (h) MHC Class II, (i) Thy-1, (j) secondary Ab only; (k) flow cytometric analysis of cells recovered from MTS20⁺/24⁺ cell graft seeded with CD4⁻8⁻ thymocytes, showing development of single- and double-positive populations. Scale bars: (a,b) 1 mm; (c-j) 100 μm. Arrows in (a,b) point to graft site, in (c,d) to filter marking graft site. Box in (c) is area shown in (e-j). Representative of 3 experiments: Grafts contained: 80000, n=1,12500, n=2, MTS20⁺/24⁺ cells; 25000, n=1, 12500, n=2, MTS20⁻/24⁻ cells.

FIG. 2 shows how MTS20⁺/24⁺ cells confer thymus function on nude mice. The graphs display a flow cytometric analysis of CD4⁺ and CD8⁺ T-cell populations in the lymph nodes of: (a,b) nude mice grafted with 500 MTS20⁺/24⁺ cells; (c,d) nude mice grafted with E12.5 whole thymus lobes; (e,f) nude mice grafted with dissociated-and-reaggregated E12.5 thymus lobes; (g,h) unmanipulated control nude mice; after gating for lymphoid cells. FIG. 2(a, c, e, g) show CD3, CD4 expression; FIG. 2(b, d, f, h) show CD3, CD8 expression. Figures refer to percentage of lymphoid cells in the upper right quadrant of each graph. Mice grafted with MTS20⁺/24⁺ cells, n=7; mice grafted with whole E12.5 thymus lobes, n=7; mice grafted with dissociated-and-reaggregated E12.5 thymus lobes, n=9; unmanipulated mice, n=15.

FIG. 3 shows (A) integration of the LoxP-flanked SV40 T antigen (Tag) plus IRES-linked selectable marker-and-stop cassette into the Foxn1 locus places SV40 Tag under control of the Foxn1 promoter and creates a Foxn1 null allele. Exon 15 is noncoding, and is spliced to the strong splice acceptor included in the SV40 Tag IRES GFP/neomycin resistance cassette. Transcription is truncated at the polyadenylation site and terminated at the transcriptional pause, thus the Foxn1 coding sequence should not be transcribed or translated. Cells in which both Foxn1 alleles are targeted are Foxn1 null and cells that would normally express Foxn1 are immortalized by SV40 Tag. The construct used for gene targeting includes an additional selectable marker under a promoter expressed in ES cells which is deleted before use of the targeted cells and is not shown. ES cells used for this targeting strategy may contain an integrated, inducible Cre transgene (not shown), or Cre may be delivered to the targeted ES cells via a viral or plasmid expression construct; and (B) After induction of Cre recombinase expression, the LoxP flanked cassette is excised by Cre-mediated recombination, leaving a single LoxP site in the intron. This restores normal Foxn1 expression, and removes SV40 Tag. Cells therefore undergo deimmortalization and are competent to differentiate upon receipt of appropriate signals.

### EXAMPLE 1

Murine TEPC Line Generation

Materials and Methods

Antibodies

The following monoclonal antibodies (mAbs) were used for immunofluorescence and flow cytometry: MTS20 and MTS24 (both rat mAbs); 4F1 (Imami, N. Dev. Immunol. 2: 161-173 (1992)); MTS10 (PharMingen); anti-cytokeratin (rabbit polyclonal anti-keratin, Dako Corporation); anti-MHC class II (M5114-biotin, PharMingen), anti-Thy-1 (T24, PharMingen); anti-CD4 (GK1.5, PE conjugated, PharMingen); anti-CD3 (14S-2C11 Cy-chrome conjugated, PharMingen); anti-CD8 (53-6.7, FITC conjugated, PharMingen).

Appropriate isotype-control antibodies (PharMingen) were used as negative controls in all experiments. Forflow cytometry, unconjugated mAbs were detected using goat anti-rat FITC (Jackson Labs) or goat anti-rabbit FITC (Sigma). For immunohistochemistry, unconjugated mAbs were detected using rabbit anti-rat HRP (Sigma), donkey anti-rabbit HRP (Diagnostics Scotland) or streptavidin FITC (Pharmingen).

Mice

Female C57BU6 and male CBA mice were caged together overnight. The morning of finding the vaginal plug was designated embryonic day 0.5 (E 0.5). Female ICRF nu/nu mice were obtained from Harlan UK and kept in ventilated, isolated cages under sterile conditions.

Histology and Immunohistochemistry

Tissues for sectioning were washed in PBS and embedded in OCT compound (Bayer Diagnostics). 8 μm frozen sections were cut onto Poly-L-lysine (Sigma) coated slides. Sections were fixed briefly in cold acetone and stained with hematoxylin and eosin. For immunohistochemical staining, sections were blocked in 10% normal serum, incubated with primary antibody for 1-2 hours followed by incubation with the appropriate secondary antibody. Additionally, some sections were then treated with tyramide amplification reagent (NEN Life Sciences) according to the manufacturer's instructions.

Flow Cytometry

Solid tissues were dissociated in 2 mg/ml hyaluronidase, 0.7 mg/ml collagenase, 0.05 mg/ml DNAse (all Sigma) at 37° C. for 30 minutes to a single cell suspension followed by extensive washing. Cells were released from lymph nodes and adult thymi by passage through a 70 μm cell strainer (Falcon). Cells were incubated with mAbs in PBS/10% FCS at 41° C. for 20 minutes and washed in PBS/FCS. For intracellular staining, cells were treated with Fix and Perm (Caltag Laboratories). Cells were analysed on a FACscan (Becton Dickinson) and data is presented using CellQuest software (Becton Dickinson).

For FACS sorting, cells were prepared as above and stained with MTS20 and MTS24 followed by anti-rat-FITC (Jackson Labs). Sorting was performed on a FACS Star (Becton Dickinson). MTS20⁺/24⁺ and MTS20⁻/24⁻ cells were collected and aliquots of the sorted cells were re-analysed by FACS. In all cases, purity of the sorted populations was greater than 95%.

Cells

Murine embryonic fibroblasts (MEF) were prepared from E13.5 or E14.5 wild-type embryos stripped of their internal organs (including thymi) and triturated to a single cell suspension. These cells were plated in DMEM (Gibco) containing 10% FCS, 50 U/ml penicillin and 50 μg/ml streptomycin, and were harvested by trypsinization (0.025% trypsin) after a minimum of 3 days. Double negative thymocytes were prepared by MACS depletion of CD4⁺ and CD8⁺ cells from thymocytes recovered from adult thymi, according to the manufacturer's instructions (Miltenyi Biotech). MTS20⁺/24⁺ and MTS20/24⁻ cells were prepared from thymi dissected from early E12.5 embryos, as above. At E12.5, each thymic lobe contains approximately 5,000 cells (unpublished data), approx. 3000 MTS20⁺/24⁺, or approx. 7000 MTS20⁻/24⁻ cells constitute the equivalent number of cells of each population to that found in two intact E12.5 thymus lobes.

Kidney Capsule Grafting

Reaggregate cultures were prepared as previously described (Anderson, G. Nature 362: 70-73 (1993)). The appropriate numbers of each cell-type were mixed in a tiny volume of medium, and the cell slurry placed in a drop on a 0.8 μm Isopore membrane filter (Millipore) floating on medium. After 24-48 hours the reaggregate was grafted under the kidney capsule of female ICRF nu/nu mice with a small piece of filter to mark the position of the graft (Hoffmann, M. W. Proc. Nati. Acad. Sci. USA 89: 2526-2530 (1992)). The following grafting conditions were us d:

Short Term Grafts

MTS20⁺/24⁺ cell graftsl2,500 cells (n=2); 80,000 cells (n=1). MTS20-124-cell graftsl2,500 cells (n=2); 25,000 cells (n=1). All with 100,000 MEF. All both with and without 100,000 CD4-8-thymocytes.

Long Term Grafts

MTS20⁺/24⁺ cell grafts 500 cells (n=1); 500 cells plus 1,000 MEF (n=1); 500 cells plus 200,000 MEF (n=2); 1,000 cells plus 1,000 MEF (n=1); 1,000 cells plus 200,000 MEF (n=1); 5,000 cells (n=1).

MTS20⁻/24⁻ cell grafts 500 cells plus 200,000 MEF (n=2); 1,000 cells plus 200,000 MEF (n=1); 10,000 cells (n=1); 10,000 cells plus 4,000 MEF (n=1); 160,000 cells plus 100,000 MEF (n=1).

Dissociated and reaggregated E12.5 thymus cell grafts 10,000 cells (n=6); 10,000 cells plus 1,000 MEF (n=2); 100,000 cells (n=1).

Unmanipulated nu/nu control mice were age matched and from the same purchase group in all experiments. Since loss of cells during experimental procedure is inevitable but not quantifiable in this model, the input cell numbers cited are overestimated.

Statistical significance was determined using the Mann-Whitney U Test.

Results

1. Generation of Functional Thymi from Populations of Isolated TEPCs

Defined numbers of purified TEPCs (MTS20⁺/24⁺) or MTS20/24⁻ cells and primary embryonic fibroblasts are mixed, reaggregated for 1-2 days in vitro, grafted under the kidney capsule of nude mice (Flanagan, S. P. Genet. Res. 8: 295 (1966); Pantelouris, E. M. Nature 217: 370-371 (1968)) and left for three weeks before analysis.

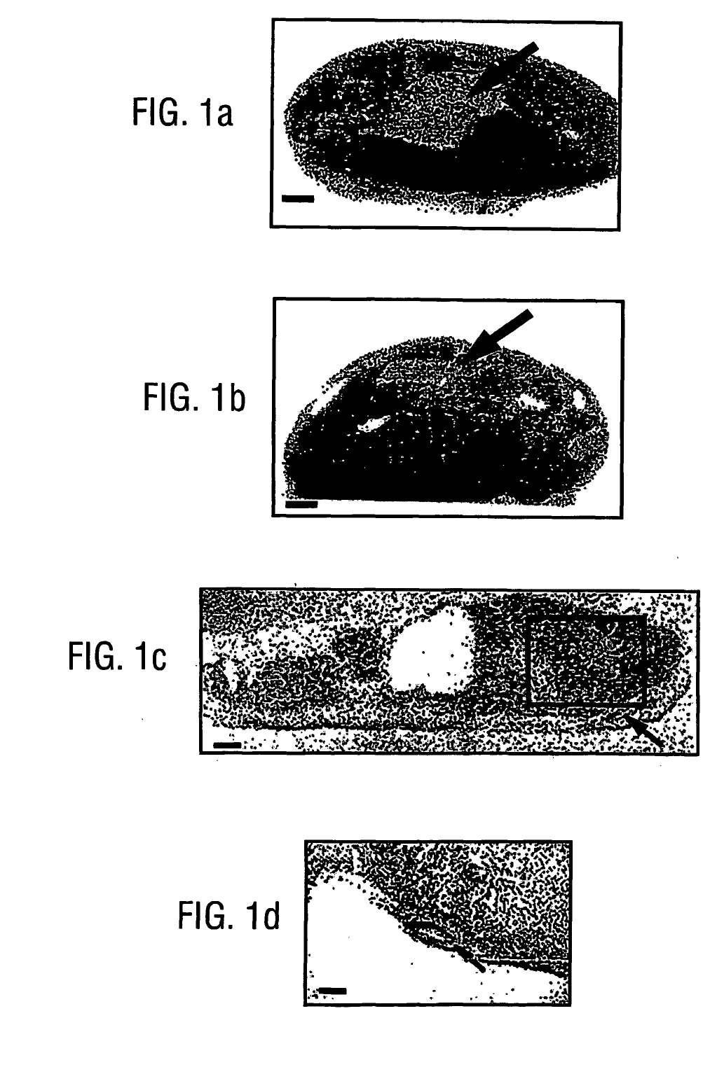

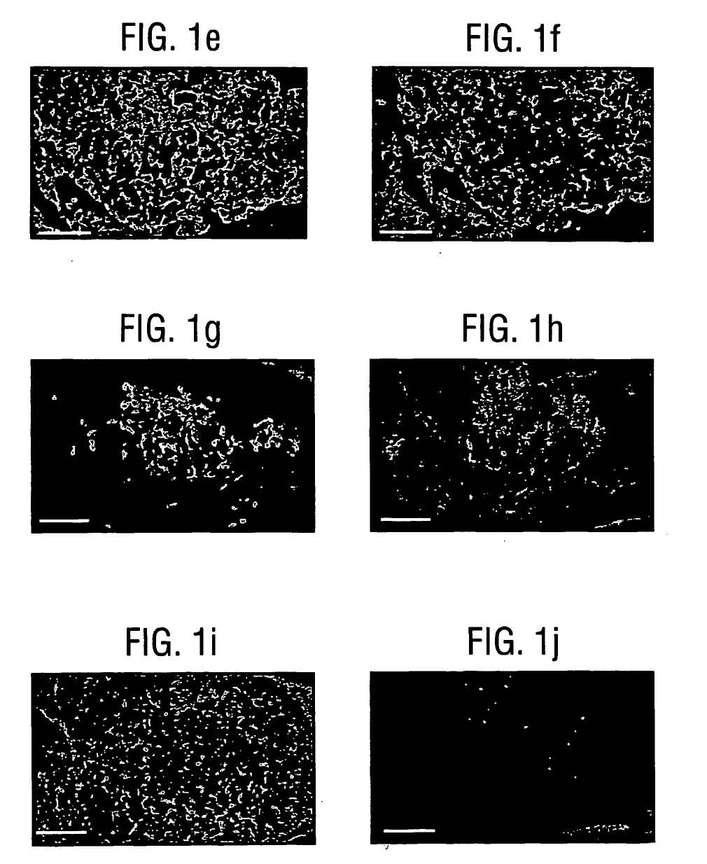

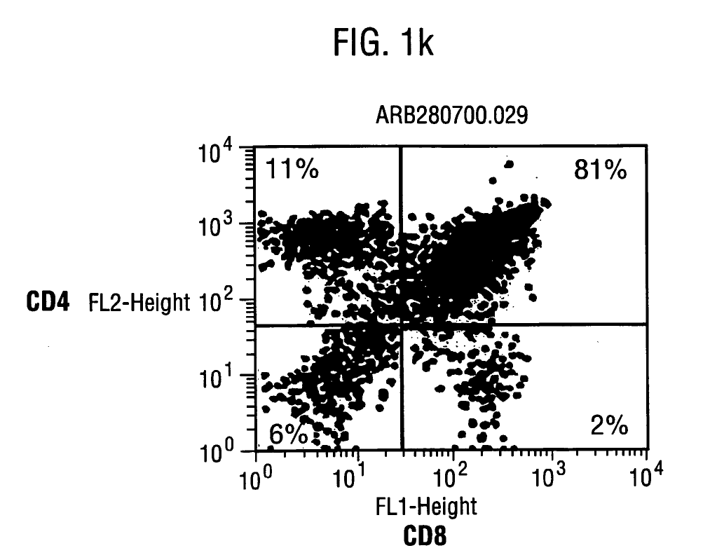

Robust grafts are recovered from all MTS20/24⁺ recipient mice (FIG. 1a). These grafts are encapsulated and vascularized, and found to contain cells of lymphoid appearance (FIG. 1c).

Immunohistochemical analysis reveals extensive networks of cytokeratin-positive epithelial cells within each graft (FIG. 1e), which mostly express MHC class 11 (FIG. 1h) and encompass both 4F1-positive and MTS10-positive areas (FIGS. 1f,g). Medullary and cortical areas are clearly visible in hematoxylin and eosin stained sections (FIG. 1c). The lymphoid cells within the grafts are Thy-1-positive (Van Ewijk, W. Eur. J. Immunol. 12: 262-271 (1982)) (FIG. 1i) and B220-negative, indicating that they are T-lineage cells. They are found mainly within the keratin positive areas. Some epithelial Thy-1 staining is also evident.

These data indicate that cells within the MTS20⁺/24⁺ population can differentiate into cells expressing markers of both mature cortical (4F1⁺) and mature medullary (MTS10⁺) thymic epithelial lineages. Furthermore, MTS20/24⁺ cells, or their progeny, can attract T-cell progenitors and initiate vascularization of the graft. Control grafts containing only primary embryonic fibroblasts survive in some recipients but are not colonized by lymphoid cells. Nor do they express keratin, 4F1 or MTS10.

In MTS20⁻/24⁻ cell recipients, no evidence is found of grafted cells although the graft sites are clearly marked in all animals (FIGS. 1a-d).

To rule out the possibility that MTS20⁻/24⁻ cells are capable of forming a functional thymus if supplied with thymocytes, as would be the case if they require thymocyte-derived factors for survival but are unable to attract T-cell progenitors, MTS20/24⁺ and MTS20⁻/24⁻ cell grafts are seeded with CD4⁻8⁻ thymocytes purified from adult thymi. Three weeks post-grafting, immunohistochemical analysis of MTS20/24⁺ grafts gives results identical to those described above. MTS20⁻/24⁻ cell grafts cannot be recovered. MTS20⁻/24⁻ cells cannot therefore reconstitute thymus function, even when supplied with immature thymocytes. These data suggest that, in addition to the roles described above, MTS20/24⁺ cells may be required directly or indirectly to support growth and survival of differentiating and/or mature cortical thymic epithelium, since the MTS20⁻/24⁻ population contains epithelial cells expressing 4F1, a marker of cortical epithelium, which has previously been thought to be sufficient to support thymocyte development to the immature CD4⁺ and CD8⁺ single positive stages (Ge, Q. Int. Immunol. 12:1127-1133 (2000); DeKoning, J. J. Immunol. 158: 2558-2566 (1997)).

To test the functional potential of the MTS20/24⁺ population, thymocyte development is analysed in grafts seeded with CD4⁻8⁻ T-cell progenitors. Flow cytometric analysis of thymocytes recovered from MTS20/24⁺ cell grafts indicate that the grafts support differentiation of CD4⁻8⁻ progenitors into CD4⁺ and CD8⁺ single-positive T-cells, the distribution of CD4⁺ and CD8⁺ subsets being identical to those within a normal adult thymus (FIG. 1k).

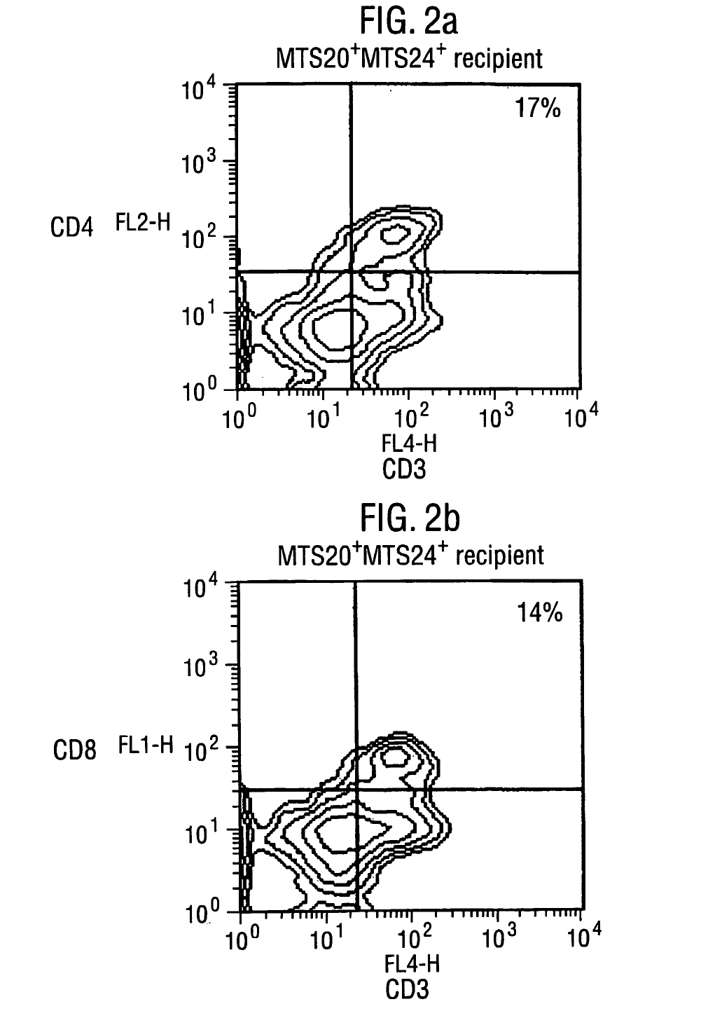

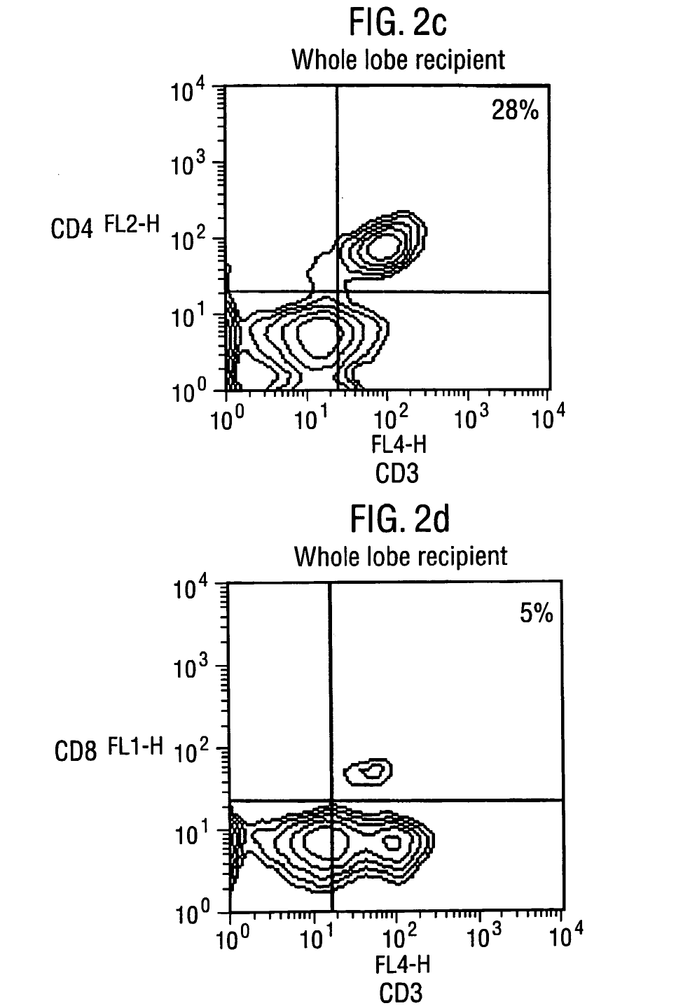

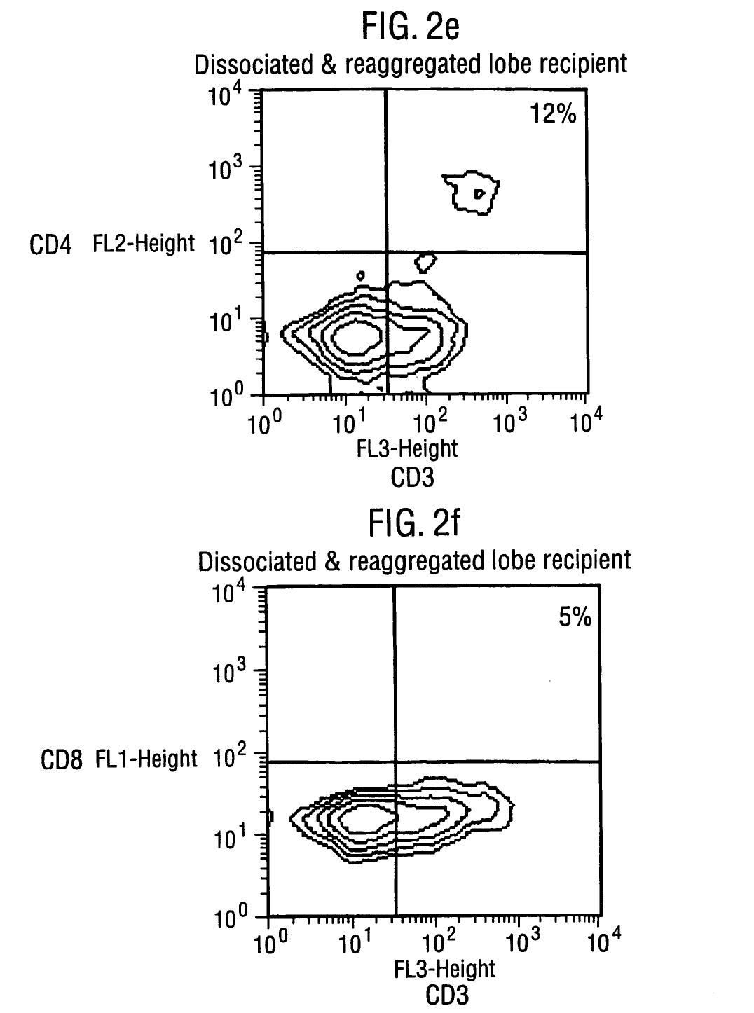

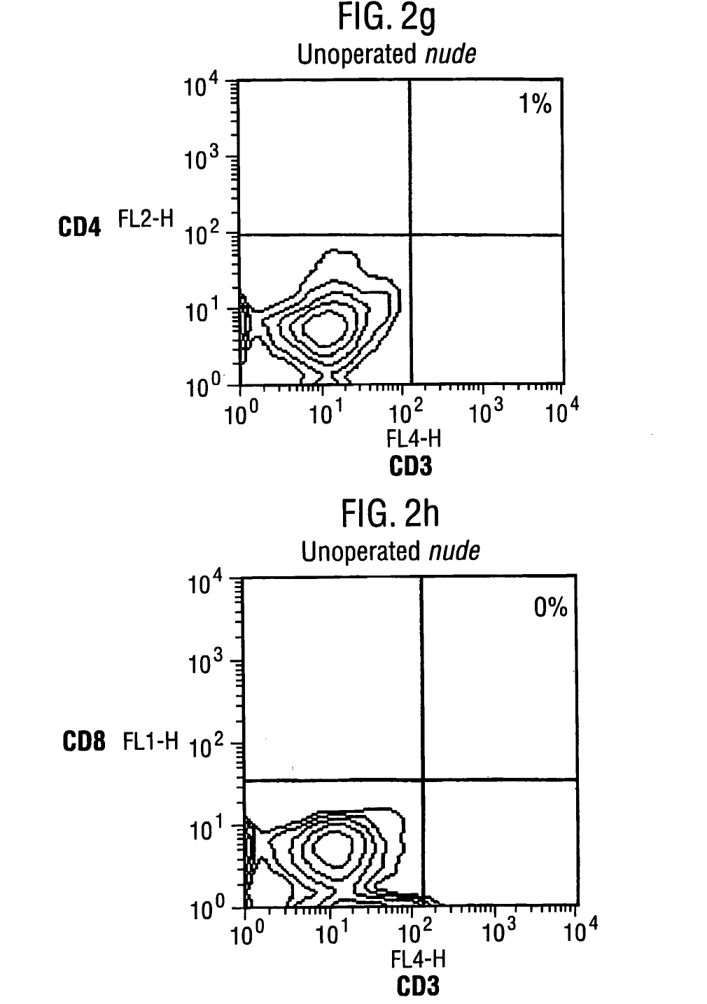

The functional potential of the MTS20/24⁺ population is further tested by assaying the presence of peripheral T-cells in recipient nude mice. In these experiments low numbers of cells are grafted under the kidney capsule of nude recipients, which are left for 12-16 weeks before analysis. Recipient mice receive grafts of 500-5,000 MTS20⁺/24⁺ cells (0.2-2 embryo-thymus equivalents per graft), 500-160,000 MTS20⁻/24⁻ cells (0.7-1.4 embryo-thymus equivalents per graft) or whole E12.5 thymic lobes. Significant CD4⁺ and CD8⁺ T-cell populations are present in the axillary, inguinal and popliteal lymph nodes of 6/7 MTS20/24⁺ cell recipient mice, including the four mice grafted with only 500 MTS20/24+cells (FIGS. 2a,b; Table 1). Equivalent populations are found in mice that receive E12.5 whole lobe grafts (FIGS. 2c,d; Table 1), whereas unmanipulated nude controls have few T-cells in these lymph nodes (FIGS. 2g,h; Table 1). Mice that receive MTS20⁻/24⁻ cells fail to gain thymus function: a distinct T-cell population is found in only 1 out of 6 recipients of MTS20⁻/24⁻ cell grafts (Table 1), and may result from growth of low numbers of contaminating MTS20/24⁺ cells since 10,000 MTS20/24⁻ cells are grafted in this instance. Mice that receive grafts ofdissociated-and-reaggregated cells from unfractionated E12.5 thymi develop peripheral T cell populations in only 5/9 cases, and do not develop CD8+T cell populations, indicating that these cells have reduced thymus-generation potential compared to MTS20⁺/24⁺ cells. Collectively, these data indicate the functional as well as phenotypic maturity of the differentiated epithelial cells within the MTS20⁺/24⁺ cell grafts. They therefore demonstrate thatthe MTS20/24⁺ cells within the E12.5 murinethymic primordium are specified TEPC which are sufficient to generate a functional thymus in vivo.

2. Improving TEPC Viability by Addition of Growth Factors

Murine TEPCs are enriched from embryonic day E12.5 thymic primordia by flow cytometry. Forward and side scatter parameters are used to select the TEPC population. Cells are re-suspended in modified DMEM (Sigma) supplemented with 2 mM glutamine, 1 mM sodium pyruvate, 0.1 mM Non-essential Amino Acids (GIBCO BRL), 50 ug/ml gentamycin solution (Sigma). Growth factors are added and the cells seeded into plates. A seeding density of approximately 4×10⁵ cells per well of a 24-well plate is used. Growth factors are added every two days. Once the TEPC cultures approach confluence, the population is diluted with supplemented DMEM (as above) and maintained in flasks at 37° C. The phenotype of these cultures is assessed periodically by immunocytochemistry, flow cytometry and gene expression analysis, to ensure maintainance of the TEPC phenotype.

3. Preparation of Immortalized TEPC Lines

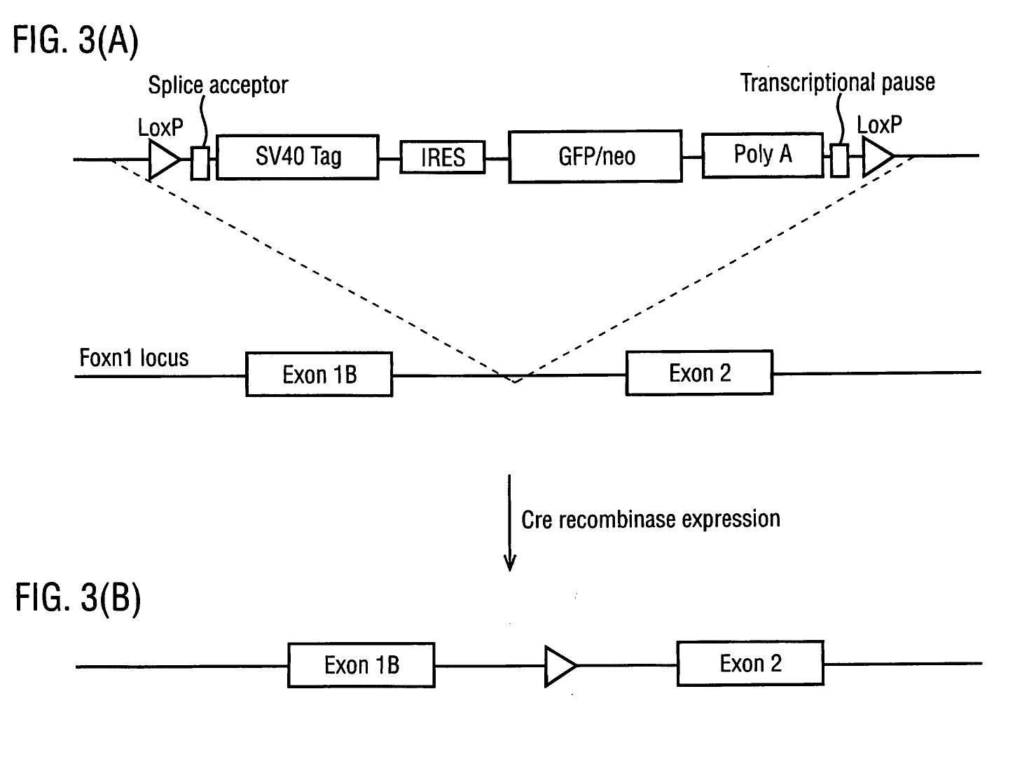

A) Transgenic ES cells are prepared in which Foxn1 expression is conditionally ablated by targeted integration of a LoxP-flanked SV40 T antigen plus selectable marker and stop cassette downstream of the Foxn1 transcriptional start (FIG. 3). Standard ES cell targeting techniques are used, and both Foxn1 alleles are targeted. As discussed elsewhere herein, thymic cells which are unable to express Foxn1 (formerly whn/Hfh11) cannot differentiate into mature thymic epithelial cell sub-populations. Suppression of Foxn1 expression thus leads to an arrest in development at the TEPC stage.

Foxn1^{-/-} ES cells are injected into wild-type blastocysts and transferred to pseudopregnantfemale mice. This creates chimaeric embryos. TEPCs carrying the LoxP-flanked SV40 T antigen/selectable marker/stop cassette are obtained from chimaeric embryonic thymi. Embryos at day E12.5 are sacrificed and their thymi removed. TEPCs are enriched by FACS purification using forward and side scatter parameters. Cells are dissociated to single cell suspension, and grown in supplemented modified DMEM. Antibiotic is added to the medium to ensure growth of only those transgenic cells in which the Foxn1 promoter is active. Candidate TEPC lines are characterized phenotypically using monoclonal antibodies MTS20 and MTS24, or other markers expressed in TEPCs as appropriate. Lines expressing TEPC markers are selected for further propagation.

B) Alternatively, Foxn1 is conditionally ablated by targeted integration of a LoxP-flanked selectable marker and stop cassette downstream of the Foxn1 transcriptional start, and a transgene containing a LoxP-flanked SV40 T antigen regulated by the Tet promoter is introduced into the same ES cells, such that SV40 T antigen expression is induced by growth of the cells in tetracyclin analogues. TEPC lines are derived as above, upon growth in medium containing a tetracyclin analogue.

C) Alternatively, Foxn1 is conditionally ablated by random integration of a transgene spanning the Foxn1 locus, in which a LoxP-flanked selectable marker and stop cassette has been placed downstream of the Foxn1 transcriptional start, and a transgene containing a LoxP-flanked SV40 T antigen regulated by the Tet promoter is introduced into the same ES cells, such that SV40 T antigen expression is induced by growth of the cells in tetracyclin analogues. Transgenic mice are produced and backcrossed onto a Foxn1^{-/-} background (i.e. nu/nu). TEPC lines are derived as above, upon growth in medium containing a tetracyclin analogue.

D) Alternatively, TEPC lines are derived by retroviral or adenoviral delivery of a conditionally inactivatable SV40 T antigen to purified TEPC or enriched TEPC cultures. Reversible immortalisation is achieved.

E) Alternatively, TEPC lines are derived by retroviral or adenoviral delivery of a conditionally inactivatable SV40 T antigen, and Foxn1-antisense oligonucleotides, to purified TEPC or TEPC-enriched cultures. Both reversible immortalisation and reversible suppression of Foxn1 are achieved.

4. Differentiation of TEPCs into Mature Thymic Epithelial Cell-Types

Reactivation of Foxn1 and/or deletion of SV40 large T antigen in TEPC cell lines prepared as in (3) above enables differentiation of the TEPCs into mature thymic epithelial cell types, including cortical and medullary thymic epithelial cells: Cre-mediated excision of the SV40 large T antigen/selectable marker/stop cassette, or the separate SV40 large T antigen- and selectable marker and stop-cassettes, is achieved by transforming the cell line with a Cre expressing vector, or by activation of an inducible Cre transgene within the TEPC.

Following reactivation of Foxn1 and deletion of SV40 large T antigen, the TEPC line is cultured in the presence of thymic mesenchymal cells and/or haematopoietic stem cells and/or lymphiod progenitor cells and/or thymocytes. Differentiation of TEPCs into cortical and medullary thymic epithelial sub-populations is assessed using immunocytochemistry and gene expression analysis. If required, undifferentiated TEPC are removed from the culture by FACS.

### EXAMPLE 2

Human TEPC Line Derivation.

Human Foxn1^{+/-} ES cell lines are generated as now described. TEPC are enriched from thymic tissue obtained from first trimester human abortuses by flow cytometry using light scatter parameters as described for mouse TEPCs, and re-suspended in D-valine-modified supplemented DMEM, and seeded into plates.

Inactivatable SV40 T antigen and reversible Foxn1 suppression is then delivered into the culture of TEPCs by addition of Foxn1-antisense oligonucleotides or Foxn1-antisense morpholino oligonucleotides to TEPC cultures, or by viral or liposomal delivery of a construct that conditionally expresses antisense-Foxn1, and by retroviral or adenoviral delivery of a conditionally inactivatable (i.e. LoxP flanked) SV40 T antigen.

Individual colonies of cells are sampled for phenotypic analysis by RT-PCR and immunohistochemistry, which assesses expression of markers characteristic of murine TEPC, including the human orthologues of MTS20 and MTS24. Candidate TEPC are selected for further propagation and cloning by phenotype, and then characterized via lineage and functional analyses after SV40 T antigen inactivation/Foxn1 activation, which is achieved by delivering Cre to the immortalised cells as above, and by withdrawing Foxn1 antisense oligonucleotides from the medium when appropriate.

Materials and Method for Demonstration of Suppression of Foxn1 Expression by Antisense Olig nucle tides

Mice

Embryos for whole embryo culture were obtained from C57BU6 females mated with heterozygous Foxn1-βgal males, which are targeted transgenic mice in which a lacZ transgene has been inserted into the Foxn1 (whn) locus such that transcription of LacZ is controlled by the Foxn1 promoter (Nehls, M. et al. Two genetically separable steps in the differentiation of thymic epithelium. Science 272, 886-889 (1996)), and were collected at day 10.5 of embryonic development (E10.5). The day of appearance of a vaginal plug was considered E0.5.

Oligonucleotides

The fluorescein-tagged Foxn1-antisense morpholino oligonucleotide and fluorescein-tagged control morpholino oligonucleotide were obtained from Genetools, LLC (Corvallis, Oreg.). The sequence of these oligos was determined according to the supplier's instructions.

Whole Embryo Culture and Electroporation of Antisense Oligonucleotides

E10.5 embryos were dissected free of uterine muscle and decidua, with the placenta intact. Each embryo was gently pushed through a small (2-3 mm) slit made in an avascular region of the yolk sac and the amnion was removed. Following dissection, embryos were placed on one side in a small drop of medium. Fluorescein-tagged Foxn1-antisense morpholino oligonucleotide (50 micromolar) or fluorescein-tagged control morpholino oligonucleotide (50 micromolar) was then microinjected into the lumen of the third pharyngeal pouch on one side of the embryo, leaving the opposite pouch as a control. Alternatively, embryos were mock injected. Electroporation (2 or 3 pulses of 30V, 50 milliseconds) using a BTX830 apparatus (Qbiogene) in conjunction with 1 mm genetrode electrodes (Qbiogene) was then used to introduce the oligonucleotides into cells of the third pharyngeal pouch. Embryos were transferred to a rotating culture system (BTC Engineering, Cambridge, UK) with one embryo in 2 ml whole embryo culture medium per bottle and a continuous supply of 95% oxygen. Embryos were monitored during the 30 hour culture period before analysis.

Xgal (5-bromo-4-chloro-indolyl-β-D-galactopyranoside) Staining of Whole Embryos.

Embryos were fixed for 30-60 minutes at 4° C. in a solution of (0.2% glutaraldehyde, 1% formaldehyde, 5 mM EDTA, 2 mM MgCl₂, 0.02% NP40) in PBS, and then washed 3 times for 5 minutes at room temperature in PBS plus 0.02% NP40. They were then stained for 4-12 hours at 37° C. in the dark in a solution of (1 mg/ml Xgal, 5 mM potassium ferrocyanide, 5 mM potassium ferricyanide, 2 mM MgCl₂, 0.02% NP40) in PBS, until staining was visible. Following staining, embryos were washed 3 times for 5 minutes at room temperature in PBS plus 0.02% NP40, and were then re-fixed for 30-60 minutes at 4° C. as above. Embryos were then washed in PBS plus 0.02% NP40, and stored at 4° C. until processing for conventional paraffin embedding and sectioning as necessary.

Histology

Fresh tissue was snap frozen in OCT Compound (Tissue TEK) and 8 micrometer transverse sections cut using a cryotome 650 (Anglia Scientific). Sections were stored at −80° C. and air-dried before use. For histological staining sections were acetone-fixed, washed and incubated in Mayer's haematoxylin (Sigma) for 10 minutes then eosin (Sigma) for 5 minutes.

Results

Evidence for the ability of antisense oligonucleotides to suppress Foxn1 expression was sought via assay of Foxn1 expression in cultured mouse embryos. Embryos obtained from the C57BL6 x Foxn1-βgal^{+/-} cross described above were dissected at E10.5, microinjected with fluorescein-tagged Foxn1-antisense morpholino oligonucleotide, or control fluorescein-tagged antisense morpholino oligonucleotide, or were mock-injected, and were then electroporated and cultured for 30 hours, as described above. At the end of the culture period, embryos were examined by microscopy, and those showing normal development were taken forward for further analysis. Initially, these embryos were genotyped by PCR. Embryos that were heterozygous for the lacZ transgene were stained with Xgal to reveal beta-galactosidase activity, which reports Foxn1 expression (Nehls 1996; Gordon, J., Gcm2 and Foxn1 mark early parathyroid- and thymus-specific domains in the developing third pharyngeal pouch. Mech Dev 103, 141-143 (2000)). Following staining, expression was scored visually and recorded photographically. Embryos were then processed for sectioning and detailed histological examination as described.

Strong Xgal staining was observed in both thymic lobes of mock-injected control embryos, and embryos injected/electroporated with the control morpholino oligonucleotide. Embryos injected/electroporated with the Foxn1-antisense morpholino oligonucleotide showed strong Xgal staining in the thymic lobe derived from the uninjected/electoporated pouch, but very weak, or no Xgal staining in the thymic lobe derived from the injected/electroporated pouch. Histological examination of sections derived from these embryos confirmed these data, and indicated that embryonic development was equivalent in unmanipulated control embryos, mock-injected embryos, and embryos injected/electroporated with the control morpholino oligonucleotide. Thus indicating that the procedure itself had no effect on Foxn1 expression, or on thymus development. Therefore, these data demonstrate that introduction of the Foxn1-antisense morpholino oligonucleotide into third pharyngeal pouch cells was able to suppress Foxn1-expression, as reported by beta-galactosidase activity.

### EXAMPLE 3

### Development of Customized Human T-Cell Repertoires.

During thymocyte development, the MHC ligands required for positive and negative selection need not be supplied by thymic epithelial cells, since fibroblasts and haematopoietic-derived cells can mediate positive selection of both CD4⁺ and CD8⁺ T-cells, while negative selection is imposed both by bone marrow-derived thymic dendritic cells (DC) and medullary epithelial cells. T-cell maturation can thus be viewed as thymus-dependent but thymic-MHC independent. Furthermore, mouse thymic epithelium can support human T-cell development.

Therefore, mouse TEPC lines are used to generate specific human T-cell repertoires via a strategy whereby epithelial function in chimaeric reaggregate foetal thymic organ cultures (RFTOCs) (Anderson, G., Jenkinson, E. J., Moore, N. C. & Owen, J. J. T. MHC class 11 positive epithelium and mesenchyme cells are both required for T-cell development in the thymus. Nature 362, 70-73 (1993)) is supplied by differentiated mouse TEPC lines, while MHC-selection is mediated by human fibroblasts and DC. Human TEPCs could similarly be used.

This approach is carried out in a fully murine system in which Cre-induced differentiating TEPC lines are supplemented with allogeneic MHC specificities. Here, fibroblasts and DC from mice haplotype-mismatched with the TEPC are included in TEPC line-based RFTOCs.

These are grafted into nude mice syngeneic with the TEPC line and left for 12-16 weeks, after which the ability of the recipient mice to reject skin grafts syngeneic and allogeneic with TEPC and “donor” haplotypes is determined. Acceptance of both TEPC and “donor” haplotype skin grafts, but rejection of 3^{rd} party grafts demonstrates that the T-cell repertoires generated in these chimaeric RFTOC grafts are fully functional and tolerant to both TEPC and donor tissues.

To generate human T cells, the system is modified such that fibroblasts and haematopoietic progenitor cells from individual humans are added to mouse TEPC-based RFTOCs. These are grafted into SCID/nude mice syngeneic with the TEPC line, which receives additional human haematopoietic progenitor cells. The specificities of the resulting human T-cell repertoires are addressed using cytotoxicity assays in which their ability to lyse syngeneic and allogeneic human and mouse cells is tested. If necessary, tolerisation of the T-cell repertoire to human proteins is optimised by injection of human cell lysates into grafted RFTOCs, or by the use of human DC elicited in vitro in the presence of human cell lysates prior to creation of the chimaeric RFTOC.

Further TEPC lines are generated from Class I- and Class II-deficient mice, since no positive or negative selection of the T-cell repertoire on mouse MHC complexes could occur in this case, and such TEPC therefore have potential for the production of human T-cells for clinical use.

Human TEPC can also be used with the following modification; RFTOCs include TEPC and haematopoietic progenitor cells from one or more individual human, and can also include fibroblasts from one or more individual human.

TABLE 1
Analysis flymph node cells from grafted nude recipients

| | | Total LN | CD4⁺ cells | CD8⁺ cells |
|---|---|---|---|---|
| | Graft | Cells | per 10³ LN | per 10³ LN |
| Graft | Success^{a} | (×10⁶) | cells [SEM] | cells [SEM] |
| E12.5 MTS20⁺/24⁺ | 6/7^{b} | 20 | 46 [9.9]^{d} | 33 [9.1]^{d} |
| cells | | | | |
| E12.5 MTS20⁻/24⁻ | 1/6^{c} | 13 | 16 [7.2]^{e} | 13 [4.0]^{e} |
| cells | | | | |
| D&R E12.5 thymus | 5/9 | 15 | 38 [10]^{f} | 7.8 [3.2]^{f} |
| cells | | | | |
| Intact thymus lobes | 7/7 | 19 | 190 [29]^{g} | 49 [14]^{g} |
| (2) | | | | |
| MEFs | 0/3 | | 11 [1] | 7.7 [1] |
| Ungrafted nu/nu | n = 15 | 24 | 10 [2.3] | 7.4 [2] |
| Ungrafted wild type | n = 12 | 8.8 | 240 [16] | 160 [15] |

^{a}Grafts were taken to have conferred thymus function where T-cell numbers in recipients exceeded two standard deviations from the mean of the ungrafted nude population.
^{b}The unsuccessful graft contained 1 × 10³ MTS20⁺/24⁺ cells.
^{c}The successful graft contained 1 × 10⁴ MTS20⁻/24⁻ cells.
^{d}MTS20⁺/24⁺ recipients versus ungrafted nude, p = 0.0006 for CD4⁺, p = 0.011 for CD8⁺.
^{e}MTS20⁻/24⁻ recipients versus ungrafted nude, p > 0.6 for CD4⁺, p > 0.2 for CD8⁺.
^{f}dissociated and reaggregated recipients versus ungrafted nude, p = 0.02 for CD4⁺, p > 0.4 for CD8⁺.
^{g}whole lobe recipients versus ungrafted nude, p = 0.0002 for CD4⁺, p = 0.010 for CD8⁺.
Statistical analys s include data from all mice in each group. SEM, standard error of the mean; D&R, Dissociated and reaggregated; LN, lymph node.

1. An in vitro method of obtaining a mature thymic epithelial cell, comprising improving the viability of a population of isolated thymic epithelial progenitor cells (TEPCs), by contacting the cells, or one or more ancestors thereof, with at least one viability promoting agent, and then allowing or causing the TEPCs to differentiate.

2. The method of claim 1, wherein the at least one viability promoting agent inhibits differentiation of the TEPCs into cortical and/or medullary thymic epithelial cells.

3. The method of claim 1, wherein the at least one viability promoting agent is selected from the group consisting of insulin-like growth factor 1 (IGF-1), epidermal growth factor (EGF), Insulin, hydrocortisone, transferrin, high density lipoprotein (HDL), bone morphogenetic protein (BMP)2, BMP4 and/or BMP7

4. The method of claim 1 or 2, wherein the at least one viability promoting agent causes a change in the genotype of a TEPC in the population.

5. The method of claim 4, wherein the at least one viability promoting agent comprises or consists of an immortalizing oncogene.

6. The method of claim 4 or 5, wherein the at least one viability promoting agent is conditionally inactivatable.

7. The method of any one of claims 4 to 6, wherein the at least one viability promoting agent causes reversible suppression of Foxn1 expression.

8. A TEPC comprising a nucleic acid construct which reversibly suppresses Foxn1 expression.

9. A TEPC line according to claim 8.

10. A pharmaceutical composition comprising the TEPC of claim 8 or 9, and a pharmaceutically acceptable vehicle, diluent or carrier.

11. The composition of claim 10, further comprising one or more agents which promote differentiation of the TEPC into a cortical or medullary thymic epithelial fate.

12. A method of restoring or enhancing thymic function in a patient, the method comprising administering to the patient a pharmaceutically effective dose of a TEPC according to claim 8 or 9, or a pharmaceutical composition according to claim 10 or 11.

13. Use of a non-human cortical and/or medullary thymic epithelial cell, obtained from a cell according to claims 8 or 9 in generation of a human T cell from a human HSC.

14. A method of obtaining mature thymic epithelial cells, comprising enriching a population of animal cells for TEPCs by maintaining a source of said animal cells under culture conditions conducive to cell survival, wherein the source of said animal cells includes animal cells containing a nucleic acid construct which inhibits differentiation of TEPCs, culturing said animal cells, and allowing or causing the TEPCs to differentiate.

15. The method of claim 14, wherein differentiation of said TEPCs is inhibited by inserting a genetic construct into said animal cells which reversibly suppress s expression of a gene essential to differentiation of TEPCs

16. An animal cell comprising at least one nucleic add construct which reversibly suppresses expression in a TEPC of a gene whose xpression is essential to differentiation of the TEPC.

17. A transgenic non-human animal which comprises a source of cells suitable for the method of claim 15.

18. A vector for use in genetically modifying cells so as to be suitable for use in the method of claim 15, comprising a sequence that combines with the genome f said animal cell so as to reversibly suppress expression of a gene essential to differentiation of a TEPC.

19. A vector according to claim 18, for homologous recombination with the genome of said animal cell.

20. A vector according to claim 19, for homologous recombination into a Foxn1 gene of an animal cell.

21. A vector according to claim 19, for homologous recombination into a Foxn1 gene of a human cell.

22. A vector according to any of claims 18 to 21 which additionally Includes recognition sequences, eg Lox P or FRT sites, which allow subsequent excision of the integrated construct via site-specific recombination.

23. Use of a TEPC according to claim 8 or 9 to assay gene function in T cell development.
